(19) United States
(12) Patent Application Publication
(10) Pub. No.: US 2020/0171170 A1
(43) Pub. Date: Jun. 4, 2020

(54) MATERIALS AND METHODS FOR INCREASING IMMUNE RESPONSES

(71) Applicant: Mayo Foundation for Medical Education and Research, Rochester, MN (US)

(72) Inventor: Larry R. Pease, Rochester, MN (US)

(73) Assignee: Mayo Foundation for Medical Education and Research, Rochester, MN (US)

(21) Appl. No.: 16/623,268

(22) PCT Filed: Jun. 15, 2018
(86) PCT No.: PCT/US2018/037874

§ 371 Date: Dec. 16, 2019

Related U.S. Application Data
(60) Provisional application No. 62/618,399, filed on Jan. 17, 2018.
(60) Provisional application No. 62/521,011, filed on Jun. 16, 2017.

(51) Int. Cl. A61K 48/00; A61K 35/761; A61K 38/17; C12N 7/00

(52) U.S. Cl. CPC A61K 48/005; A61K 35/761; C12N 2740/15043; C12N 7/00; C12N 2710/10343; A61K 38/1774

(57) ABSTRACT

This document relates to materials and methods for activating naïve T cells in vivo. For example, methods of activating naïve T cells in vivo to treat cancer are provided.

### CROSS-REFERENCE TO RELATED APPLICATIONS

This application claims priority to U.S. Application Ser. No. 62/618,399, filed on Jan. 17, 2018, and 62/521,011, filed on Jun. 16, 2017. The disclosures of the prior applications are considered part of the disclosure of this application, and are incorporated in their entirety into this application.

### BACKGROUND

### 1. Technical Field

This document relates to materials and methods for activating naïve T cells in vivo. For example, in vivo activation of naïve T cells can be used to target cells (e.g., cancer cells) expressing a tumor antigen (e.g., a tumor-specific antigen).

### 2. Background Information

Approximately 22,000 people die from cancer each day globally. Cancers infiltrated by CD8+ T cells tend to have better prognoses than those devoid of these immune cells. However, effective antitumor cellular immunity is limited by the available T-cell receptor (TcR) repertoire consisting primarily of low affinity receptors specific for tumor associated antigens.

### SUMMARY

This document provides materials and methods for activating naïve T cells (e.g., naïve T cells expressing tumor antigen receptors) in vivo. For example, naïve T cells expressing tumor-specific antigen receptors can be activated (e.g., to become cytotoxic T lymphocytes (CTLs)) in vivo by encountering antigens (e.g., antigens presented on an antigen presenting cell (APC) such as a subcapsular sinus macrophage and/or a dendritic cell) in a lymph node. The in vivo activated T cells can target cells (e.g., cancer cells) presenting the antigen (e.g., a tumor antigen) recognized by the tumor-specific antigen receptors. In some cases, the in vivo activated T cells can be expanded in vivo. Also provided herein are methods for using in vivo activation of naïve T cells as described herein (e.g., by in vivo activation of naïve T cells expressing tumor-specific antigen receptors). For example, in vivo activation of naïve T cells as described herein can be used to treat mammals (e.g., humans) having cancer.

As demonstrated herein, adoptively transferred naïve CD8+ T cells can migrate to a lymph node where they can encounter a virus (e.g., an adenovirus) encoding an allogeneic major histocompatibility complex class I (MHC I) antigen that can activate the naïve CD8+ T cells in vivo. Having the ability to activate naïve T cells expressing antigen receptors (e.g., tumor-specific antigen receptors) in vivo provides a unique and unrealized opportunity to generate CTLs capable of targeting (e.g., locating and destroying) cells (e.g., cancer cells) expressing a tumor antigen (e.g., a tumor-specific antigen) that can be recognized by the antigen receptor. For example, the ability to activate naïve T cells expressing tumor-specific antigen receptors in vivo provides the opportunity to target cancer cells, including cancer cells in solid tumors, that are otherwise undetectable by the immune system (e.g., cancers including quiescent cancer cells and/or cancers having escaped chemotherapy). In addition, the materials and methods described herein can be more conducible to “off the shelf” reagents. As such, personalized therapies in the form of tumor-specific immune responses can be rapidly and efficiently applied to wide patient populations while limiting costs.

As also described herein, using a virus (e.g., an adenovirus) designed to express an MHC I polypeptide (e.g., an allogeneic MHC I polypeptide) to activate naïve T cells within a mammal can result in the activation of many different naïve T cells within the mammal, thereby producing a polyclonal T cell response in the mammal. In some case, a virus (e.g., an adenovirus) designed to express an MHC I polypeptide (e.g., an allogeneic MHC I polypeptide) can be used to activate more than 1, 2.5, 5, 10, 15, or 20 percent of the naïve T cells within a mammal or can be used to activate more than 1, 2.5, 5, 10, 15, or 20 percent of the naïve T cells within a lymph node of a mammal. In addition, the CD8⁺ T cells that are activated in vivo using a virus (e.g., an adenovirus) designed to express an MHC I polypeptide (e.g., an allogeneic MHC I polypeptide) can be potent killers of target cells recognized by those activated CD8⁺ T cells. This level of target cell killing can be greater than that observed by comparable CD8⁺ T cells that are activated in vitro.

As further described herein, the naïve T cells that are activated using a virus (e.g., an adenovirus) designed to express an MHC I polypeptide (e.g., an allogeneic MHC I polypeptide) as described herein can be engineered (e.g., engineered in vivo or in vitro) to express an antigen receptor to a desired target before (or, for in vivo approaches, after or at the same time as) being activated. For example, when engineering naïve T cells in vivo, a vector (e.g., a viral vector such as a lentiviral vector or retroviral vector) encoding an antigen receptor (e.g., a chimeric antigen receptor such as a chimeric antigen receptor specific for a tumor antigen) can be administered to the mammal (e.g., a human) before the mammal is administered a virus (e.g., an adenovirus) designed to express an MHC I polypeptide (e.g., an allogeneic MHC I polypeptide), after the mammal is administered a virus (e.g., an adenovirus) designed to express an MHC I polypeptide (e.g., an allogeneic MHC I polypeptide), or at the same time that the mammal is administered a virus (e.g., an adenovirus) designed to express an MHC I polypeptide (e.g., an allogeneic MHC I polypeptide). In some cases, when engineering naïve T cells in vivo, a vector (e.g., a viral vector such as a lentiviral vector or retroviral vector) encoding an antigen receptor (e.g., a chimeric antigen receptor such as a chimeric antigen receptor specific for a tumor antigen) can be administered to the mammal (e.g., a human) before and after the mammal is administered a virus (e.g., an adenovirus) designed to express an MHC I polypeptide (e.g., an allogeneic MHC I polypeptide). In some cases, when engineering naïve T cells in vivo, a vector (e.g., a viral vector such as a lentiviral vector or retroviral vector) encoding an antigen receptor (e.g., a chimeric antigen receptor such as a chimeric antigen receptor specific for a tumor antigen) can be administered to the mammal (e.g., a human) before, after, and at the same time the mammal is administered a virus (e.g., an adenovirus) designed to express an MHC I polypeptide (e.g., an allogeneic MHC I polypeptide).

In cases when naïve T cells are engineered in vitro, a vector (e.g., a viral vector such as a lentiviral vector or retroviral vector) encoding an antigen receptor (e.g., a chimeric antigen receptor such as a chimeric antigen receptor specific for a tumor antigen) can be introduced into in vitro naïve T cells obtained from a mammal (e.g., a human) and introduced back into that mammal before that mammal is administered a virus (e.g., an adenovirus) designed to express an MHC I polypeptide (e.g., an allogeneic MHC I polypeptide). In some cases, the in vitro naïve T cells can be treated with one or more agents designed to stimulate the cells (e.g., anti-CD3 agents, anti-CD38 agents, interleukin (IL) 2, IL15, or combinations thereof) before, after, or both before and after the vector is introduced into the cells.

When applying the methods and materials described herein specifically to humans or human cells, the MHC I polypeptides described herein can be referred to as HLA polypeptides (e.g., HLA-A, HLA-B, and/or HLA-C polypeptides) or human MHC I polypeptides.

In general, one aspect of this document features a method for activating a naïve T cell in a mammal. The method includes, or consists essentially of, engineering a naïve T cell to express an antigen receptor, thereby forming an engineered naïve T cell, and activating the engineered naïve T cell in the mammal. The mammal can be a human. The naïve T cell can be a naïve cytotoxic T lymphocyte. The antigen receptor can be a chimeric antigen receptor. The antigen receptor can be a tumor-specific or antigen receptor. In some cases, the engineering can include ex vivo engineering. The ex vivo engineering can include obtaining the naïve T cell from the mammal, introducing nucleic acid encoding the antigen receptor into the naïve T cells to produce the engineered naïve T cell, and administering the engineered naïve T cells to the mammal. The introducing can include transducing the naïve T cells with a viral vector encoding the antigen receptor. The viral vector can be a lentiviral vector or a retroviral vector. The administering can include intravenous injection. In some cases, the engineering can include in situ engineering. The in situ engineering can include administering a viral vector encoding the antigen receptor to the mammal. The administering can include intradermal injection. The intradermal injection can be directly into a lymph node. The viral vector can be an adenoviral vector. The activating the engineered naïve T cell in vivo can include administering a viral vector encoding an antigen to the mammal. The antigen can be an alloantigen. The alloantigen can be an allogeneic major histocompatibility complex class I antigen. The viral vector can be an adenoviral vector. The administering can include intradermal injection. The intradermal injection can be directly into a lymph node.

In another aspect, this document features a method for treating a mammal having cancer. The method includes, or consists essentially of, engineering a naïve T cell to express a tumor-specific antigen receptor, thereby forming an engineered naïve T cell, and activating the engineered naïve T cell in vivo. The mammal can be a human. The cancer can be acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMOL)), Hodgkin's lymphoma, non-Hodgkin's lymphoma, myelomas, ovarian cancer, breast cancer, prostate cancer, or colon cancer. The cancer can include cancer cells expressing a tumor-specific antigen. The naïve T cell can be engineered to express a tumor-specific antigen receptor that targets the tumor-specific antigen. The tumor-specific antigen can be mucin 1 (MUC-1), human epidermal growth factor receptor 2 (HER-2), or estrogen receptor (ER). In some cases, the engineering can include ex vivo engineering. The ex vivo engineering can include obtaining the naïve T cells from the mammal, introducing nucleic acid encoding the antigen receptor into the naïve T cells to produce the engineered naïve T cell, and administering the engineered naïve T cells to the mammal. The introducing can include transducing the naïve T cells with a viral vector encoding the antigen receptor. The viral vector can be a lentiviral vector. The administering can include intravenous injection. The administering can include administering from about 200 to about 1500 engineered naïve T cells (e.g., about 300 engineered naïve T cells) to the mammal. In some cases, the engineering can include in situ engineering. The in situ engineering can include administering a viral vector encoding the antigen receptor to the mammal. The administering can include intradermal injection. The intradermal injection can be directly into a lymph node. The viral vector can be an adenoviral vector. The activating the engineered naïve T cell in vivo can include administering a viral vector encoding an antigen to the mammal. The antigen can be an alloantigen. The alloantigen can be an allogeneic major histocompatibility complex class I antigen. The viral vector can be an adenoviral vector. The administering can include intradermal injection. The intradermal injection can be directly into a lymph node. The cancer can include solid tumors. The cancer can be in remission. The cancer can include quiescent cancer cells. The cancer can include cancer cells that escaped chemotherapy or are non-responsive to chemotherapy.

In another aspect, this document features a method for obtaining an activated T cell within a mammal where the activated T cell includes a heterologous antigen receptor. The method includes, or consists essentially of, (a) introducing nucleic acid encoding a heterologous antigen receptor into T cells obtained from a mammal in vitro to obtain engineered T cells, (b) administering the engineered T cells to the mammal, and (c) administering a virus including nucleic acid encoding an MHC class I polypeptide to the mammal; where an engineered T cell of the engineered T cells administered to the mammal in step (b) is activated. The mammal can be a human. The T cells obtained from the mammal can be naïve T cells. The naïve T cells can be naïve cytotoxic T lymphocytes. The antigen receptor can be a chimeric antigen receptor. The antigen receptor can be a tumor-specific antigen receptor. The nucleic acid encoding the heterologous antigen receptor can be introduced into the T cells with a viral vector comprising the nucleic acid. The viral vector can be a lentiviral vector. The engineered T cells can be administered to the mammal via intravenous injection. The engineered T cells can be administered to the mammal via injection into a lymph node of the mammal. The virus can be an adenovirus or a rhabdovirus. The virus can be administered to the mammal via intradermal injection. The virus can be administered to the mammal via direct administration into a lymph node of the mammal. The MHC class I polypeptide can be an allogeneic MHC class I polypeptide. The MHC class I polypeptide can be an HLA-A, HLA-B, or HLA-C polypeptide. The engineered T cell activated within the mammal in step (c) can include a native T cell receptor. Step (c) can activate a plurality of engineered T cells within the mammal. The activated T cells of the plurality of engineered T cells can include different native T cell receptors.

In another aspect, this document features a method for obtaining an activated T cell within a mammal where the activated T cell includes a heterologous antigen receptor. The method includes, or consists essentially of, administering to a mammal (a) nucleic acid encoding a heterologous antigen receptor and (b) a virus comprising nucleic acid encoding an MHC class I polypeptide, where the nucleic acid is introduced into T cells within the mammal to form engineered T cells including the heterologous antigen receptor, where administration of the virus activated T cells within the mammal, and where at least one T cell within the mammal includes the heterologous antigen receptor and is activated. The mammal can be a human. The at least one T can be a cytotoxic T lymphocyte. The antigen receptor can be a chimeric antigen receptor. The antigen receptor can be a tumor-specific antigen receptor. The nucleic acid encoding the heterologous antigen receptor can be introduced into the T cells with a viral vector including the nucleic acid. The viral vector can be a lentiviral vector or retroviral vector. The nucleic acid can be administered to the mammal via intravenous injection. The nucleic acid can be administered to the mammal via injection into a lymph node of said mammal. The virus can be an adenovirus or a rhabdovirus. The virus can be administered to the mammal via intradermal injection. The virus can be administered to the mammal via direct administration into a lymph node of the mammal. The nucleic acid can be administered to the mammal before the virus is administered to the mammal. The nucleic acid encoding the heterologous antigen receptor can be introduced into the T cells with a lentiviral vector including the nucleic acid. The nucleic acid can be administered to the mammal after the virus is administered to the mammal. The nucleic acid encoding the heterologous antigen receptor can be introduced into the T cells with a retroviral vector including the nucleic acid. The MHC class I polypeptide can be an allogeneic MHC class I polypeptide. The MHC class I polypeptide can be an HLA-A, HLA-B, or HLA-C polypeptide. The at least one T cell can include a native T cell receptor. The at least one T cell can be a plurality of activated T cells including the heterologous antigen receptor. The activated T cells of the plurality of the activated T cells can include different native T cell receptors.

In another aspect, this document features an isolated virus including nucleic acid encoding an MHC class I polypeptide. The virus can be a picomavirus, an adenovirus, or a rhabdovirus (e.g., a vesicular stomatitis virus). The virus can be replication-defective. The MHC class I polypeptide can be a human MHC class I polypeptide. The MHC class I polypeptide can include the amino acid sequence set forth in SEQ ID NO:4.

In another aspect, this document features a kit having a first container including a first virus including nucleic acid encoding an antigen receptor and a second container including a second virus including nucleic acid encoding an MHC class I polypeptide. The first virus can be a lentivirus or a retrovirus. The antigen receptor can be a chimeric antigen receptor. The second virus can be a picomavirus, an adenovirus, or a rhabdovirus (e.g., a vesicular stomatitis virus). The second virus can be replication-defective. The MHC class I polypeptide can be a human MHC class I polypeptide. The MHC class I polypeptide can include the amino acid sequence set forth in SEQ ID NO:4.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 shows an exemplary scheme for in vivo activation of naïve T cells expressing surrogate antigen receptors. (1) Isolated CD8+ T cells are transduced with lentivirus or retrovirus encoding surrogate receptors are adoptively transferred intravenously back into a host (bottom), or T cells are transduced in situ in draining lymph nodes (top). (2) Allogeneic MHC I (allo-MHC I) is expressed by adenovirus introduced intradermally. (3) Transduced T cells migrate into lymph nodes and encounter APC expressing allo-MHC I. (4) Allo-reactive CTLs are activated, and (5) leave lymph node and destroy cells expressing antigens targeted by surrogate receptors.

FIGS. 2A and 2B shows that normal tissue was targeted and destroyed by virus activated tissue-specific CTL. 1200 OT-1 T cells were adoptively transferred into RIP-OVA mice, then activated with TMEV-OVA. FIG. 2A contains photographs of haemotoxylin and Eosin (H&E) staining and immunohistochemistry (IHC) staining for insulin showing pancreatic inflammation within 5 days of CTL induction by virus. FIG. 2B contains a graph showing significant destruction of islets at day 21 in surviving mice. No virus was detected in pancreas by PCR. The pancreas was totally destroyed with increased numbers of OT-1 cells. Similar results were observed when replication defective adenovirus encoding ovalbumin was used to induce pancreas destruction by OT-1 T cells.

FIGS. 3A-3C are photographs of fluorescent microscopy showing transduction of lymph node (LN) cells. mTmG-mice were infected by intradermal infection with an adenovirus expressing a cre recombinase (adeno-cre). FIG. 3A shows that adeno control virus infected LN cells. FIG. 3B shows that the adeno-cre infected LN and expressed cre recombinase in the LN. FIG. 3C shows a low magnification view of LN showing marginal location of transduced cells.

FIG. 4A is a schematic of an exemplary replication-defective adenovirus (serotype 6) vector expressing a mutant MHC molecule, which functions as a universal alloantigen. FIG. 4B is a generic version of the vector construct, by using an engineered mutant MHC molecule, the MHC can be universally allogeneic to any person. Alternatively, by using a naturally occurring MHC class I molecule, the MHC can be allogeneic to a cohort or subset of a population.

FIG. 5 contains dot plots showing that allo-reactive CTLs were generated in response to adenovirus encoding allogeneic MHC I. Allo-MHC I adenovirus was introduced into LN by intradermal injection. Four days later, syngeneic (BALB/c), allogeneic (B6), and third party (C3H) labeled target cells were adoptively transferred intravenously into challenged hosts in an in vivo CTL assay. Four hours later spleen cells were harvested and analyzed by flow cytometry for the presence of introduced target cells. B6 target cells (targets expressing introduced allo-MHC I) were completely eliminated in vivo.

FIG. 6 contains dot plots showing that adoptively transferred CD8+ T cells responded to adeno-alloMHCI. Freshly isolated syngenic CD8+ T cells were labeled with carboxyfluorescein succinimidyl ester (CFSE) before transfer, followed by challenge with adeno-allo-MHC I or control virus. FIGS. 6A and 6C show that adoptively transferred CFSE-labeled T cells migrate to the LN where they encounter and respond to transduced allo-MHC I molecules. FIG. 6C also shows that the stimulated cells proliferate when stimulated with allo-MHC I, diluting the CFSE. FIGS. 6B and 6D shows that the CFSE-dilute population displayed a more activated phenotype expressing high CD44 and PD-1 (D) relative to the CFSE-dilute cells isolated from lymph nodes challenged with control adenovirus (B).

FIG. 7 is a photograph of fluorescent microscopy showing lentivirus transduction of naïve CD8+ spleen cells from a mTmG-reporter mouse. CD8+ enriched naïve spleen cells were transduced with lentivirus-cre. The cells were subsequently activated with anti-CD3/CD28+IL-2 to maintain viability in culture for 4 days. Successful transduction results in the transition from red to green fluorescence.

FIG. 8 is a dot plot showing successful in situ introduction of transgene into activated lymph node cells. Adenoviral vector encoding alloMHC was injected intradermally into mTmG reporter mice to stimulate draining lymph node, four days later lentivirus-cre was directly injected into the enlarged lymph node. After 24 hours, CD8+ T cells from the lymph node were harvested and cultured for 3 days in the presence of IL2+IL7 to allow membrane eGFP expression.

FIG. 9 contains dot plots showing successful transduction of transgene into human cells. Human CAR lentiviral vector effective at transducing human, but not mouse T cells.

FIG. 10 contains photographs showing intradermal introduction of non-replicating virus. Hu-NSG mice lack lymph nodes. Evans blue injected intradermally in the tail to mark inguinal lymph node in WT, NOD Scid IL-2Rγ^{-/-} (NSG), and human CD34+ hematopoietic cell reconstituted NSG mice (hu-NSG).

FIG. 11 contains dot plots showing alternative routes of administration for in vivo CTL. All three immunization routes were effective as indicated by the relative depletion of the B6 target cells.

FIG. 12 shows an exemplary scheme for using hu-NSG hosts. (1) Human B cells circulating in the hu-NSG host are assessed. (2) T cells from the spleen of the nu-NSG host are contacted with a lentivirus encoding a target antigen, and injected intravenously into the nu-NSG host mouse. Replication defective adenovirus 6 encoding the MHC allogeneic antigen H-2K^{b} are injected intravenously and an identical dose was injected intraperitoneally. (3) 1 week after treatment, the composition of human B cells in the blood is assessed.

FIG. 13 contains graphs showing human leukocyte composition prior to experiment of hu-NSG mice.

FIG. 14 contains graphs showing in vivo CTL activates human immune cells in hu-NSG hosts. The expected 1:1 ratio of recovered target cells was altered in all three recipients indicating a preferential killing of the K^{b+} spleen cells (panel A). The ratio of recovered K^{b+} cells was significantly lower relative to the K^{b-} target cells (panel B).

FIG. 15 contains a graph showing raw data of the drop in B cell numbers in hu-NSG mice receiving CART treatment and AD6 vaccination.

FIG. 16 contains graphs showing normalized change in CD19+ B cells following introduction of Ad6-alloMHC (K^{b}) and lenti-CAR19 transduced spleen cells from hu-NSG mice reconstituted with CD34+ cells from the identical human donor. ^{a}Statistical evaluation normalized to account for the depletion of peripheral blood cell populations in the mice caused by repetitive blood sampling. *Increase in T cells following therapy is consistent with previous CART therapy findings.

FIG. 17 contains a sequence listing of a nucleic acid sequence (SEQ ID NO:1) encoding a human MHC I polypeptide (an HLA-B40:28) and the amino acid sequence (SEQ ID NO:3) of that human MHC I polypeptide, and a sequence listing of a nucleic acid sequence (SEQ ID NO:2) encoding a human MHC I polypeptide (an HLA-DRB1*12:01:01:01) and the amino acid sequence (SEQ ID NO:4) of that human MHC I polypeptide.

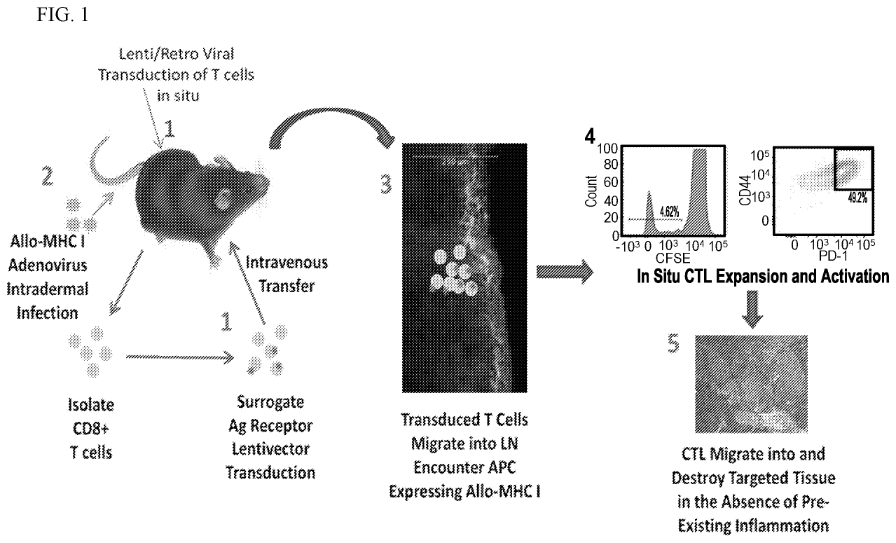

### DETAILED DESCRIPTION

This document provides materials and methods for activating naïve T cells (e.g., naïve T cells expressing tumor-specific antigen receptors) in vivo (e.g., making in vivo activated CTLs). For example, naïve T cells expressing tumor-specific antigen receptors can be activated (e.g., to become CTLs) in vivo by encountering antigens (e.g., antigens presented on an APC such as a subcapsular sinus macrophage and/or a dendritic cell) in a lymph node. In vivo activated CTLs can include effector T cells and/or memory T cells. In some cases, naïve T cells can be engineered to express tumor-specific antigen receptors ex vivo. For example, naïve T cells can be obtained, engineered ex vivo to express tumor-specific antigen receptors, and administered (e.g., by adoptive transfer) to a mammal. Adoptively transferred naïve T cells can migrate to one or more lymph nodes to be activated in vivo. In some cases, naïve T cells can be engineered to express tumor-specific antigen receptors in situ. For example, expression vectors (e.g., viral vectors) can be injected into secondary lymphoid organs such that naïve T cells are engineered in situ to express tumor-specific antigen receptors. When the naïve T cells expressing tumor-specific antigen receptors encounter an antigen (e.g., an antigen presented by an APC such as a subcapsular sinus macrophage and/or a dendritic cell), the naïve T cells are activated (e.g., to become CTLs) in vivo. The in vivo activated T cells can target cells (e.g., cancer cells) expressing the antigen (e.g., a tumor antigen) recognized by the tumor-specific antigen receptors. In some cases, the in vivo activated T cells can target cancer cells in tissues that lack current and/or preexisting inflammation. In some cases, the in vivo activated T cells do not target normal (e.g., healthy a non-cancerous) cells.

A naïve T cell that can be activated in vivo as described herein can be any appropriate naïve T cell. Examples of naïve T cells include, without limitation, CTLs (e.g., CD4+ CTLs and/or CD8+ CTLs). For example, a naïve T cell that can be activated in vivo as described herein can be a CD8+ CTL. In some cases, one or more naïve T cells can be obtained from a mammal (e.g., a mammal having cancer). For example, naïve T cells can be obtained from a mammal to be treated with the materials and method described herein.

A naïve T cell activated in vivo as described herein can express (e.g., can be engineered to express) any appropriate antigen receptor. In some cases, an antigen receptor can be a heterologous antigen receptor. In some cases, an antigen receptor can be a chimeric antigen receptor (CAR). In some cases, an antigen receptor can be a tumor antigen (e.g., tumor-specific antigen) receptor. For example, a naïve T cell can be engineered to express a tumor-specific antigen receptor that targets a tumor antigen (e.g., a cell surface tumor antigen) expressed by a cancer cell in a mammal having cancer. In some cases, an antigen receptor can be an indirect antigen receptor. For example, a naïve T cell can be engineered to express an indirect antigen receptor that targets a first antigen (e.g., an exogenous antigen). In some cases, a target cell (e.g., a cancer cell in a mammal having cancer) can express a first antigen (e.g., a tumor antigen) can be recognized by a reagent (e.g., an antibody) containing a second antigen, and a naïve T cell can be engineered to express an antigen receptor that targets the second antigen. In some cases, a tumor antigen can be a tumor-specific antigen (TSA; e.g., a tumor antigen present only on tumor cells). In some cases, a tumor antigen can be a tumor-associated antigen (TAA; e.g., an abnormal protein present on tumor cells). Examples of tumor antigens that can be recognized by a tumor antigen receptor expressed in a naïve T cell include, without limitation, mucin 1 (MUC-1), human epidermal growth factor receptor 2 (HER-2), estrogen receptor (ER), epidermal growth factor receptor (EGFR), folate receptor alpha, and mesothelin. As described herein, a naïve T cell can be engineered to have an antigen receptor (e.g., a heterologous antigen receptor) that recognizes any appropriate antigen. In some cases, a naïve T cell can be engineered to have an antigen receptor (e.g., a heterologous antigen receptor) that recognizes persistent virus antigens or senescent cells.

Any appropriate method can be used to express an antigen receptor on a naïve T cell. For example, a nucleic acid encoding an antigen receptor can be introduced into the one or more naïve T cells. In some cases, viral transduction can be used to introduce a nucleic acid encoding an antigen receptor into a non-dividing cell. A nucleic acid encoding an antigen receptor can be introduced in a naïve T cell using any appropriate method. In some cases, a nucleic acid encoding an antigen receptor can be introduced into a naïve T cell by transduction (e.g., viral transduction using a retroviral vector or a lentiviral vector) or transfection. In some cases, a nucleic acid encoding an antigen receptor can be introduced ex vivo into one or more naïve T cells. For example, ex vivo engineering of naïve T cells expressing an antigen receptor can include transducing isolated naïve T cells with a lentiviral vector encoding an antigen receptor. In cases where naïve T cells are engineered ex vivo to express an antigen receptor, the naïve T cells can be obtained from any appropriate source (e.g., a mammal such as the mammal to be treated or a donor mammal, or a cell line). In some cases, a nucleic acid encoding an antigen receptor can be introduced into one or more naïve T cells in situ into the lymphatic system (e.g., into one or more secondary lymphoid organs such as the lymph nodes and the spleen). For example, in situ engineering of naïve T cells to express an antigen receptor can include intradermal (ID) injection (e.g., directly into one or more lymph nodes) of a lentiviral vector encoding an antigen receptor.

Any appropriate method can be used to activate the naïve T cells described herein (e.g., engineered naïve T cells such as naïve T cells designed to express tumor-specific antigen receptors). For example, naïve T cells expressing tumor-specific antigen receptors can be activated in vivo by administering one or more immunogens (e.g., antigens) to a mammal. Any appropriate immunogen can be used to activate a naïve T cell described herein. In some cases, an immunogen can be a cell surface antigen (e.g., a cell surface antigen expressed by a cancer cell). In some cases, an immunogen can be an allogeneic immunogen (e.g., an allogeneic antigen (also referred to as an alloantigen)). Examples of antigens that can be used to activate the naïve T cells described herein include, without limitation, an allogeneic MHC class I polypeptide (allo-MHC I or alloMHC I polypeptide) and an allogeneic MHC class II polypeptide (allo-MHC II or alloMHC II polypeptide). Such antigens can be presented as one or more fragments in the context of an MHC molecule such as MHC I. For example, naïve T cells expressing tumor-specific antigen receptors can be activated in vivo by administering allo-MHC I to a mammal.

Any appropriate method can be used to administer an immunogen (e.g., an antigen) to a mammal (e.g., a human). Examples of methods of administering immunogens to a mammal can include, without limitation, injections (e.g., intravenous (IV), ID, intramuscular (IM) injection, or subcutaneous). In some cases, an antigen can be encoded by a vector (e.g., a viral vector), and the vector can be administered to a mammal.

An exemplary nucleic acid sequence encoding a human allo-MHC I can include a sequence as set forth in SEQ ID NO: 1. Nucleic acid encoding a human MHC I polypeptide (e.g., an HLA-A polypeptide, an HLA-B polypeptide, or an HLA-C polypeptide) can be included within a viral vector such that cells infected with the viral vector express the encoded MHC I polypeptide. In some cases, a nucleic acid sequence encoding a human allo-MHC I can be as described elsewhere (see, e.g., Pimtanothai et al., 2000 Human Immunology 61:808-815). In some cases, a nucleic acid sequence encoding a human allo-MHC I can be as set forth in a database such as the National Center for Biotechnology Information (see, e.g., GenBank® accession numbers M84384.1, AF181842, and AF181843).

SEQ ID NO: 1
ATGCGGGTCACGGCGCCCCGAACCCTCCTCCTGCTGCTCTGGGGGGCAG
TGGCCCTGACCGAGACCTGGGCTGGCTCCCACTCCATGAGGTATTTCCA
CACCTCCGTGTCCCGGCCCGGCCGCGGGGAGCCCCGCTTCATCACCGTG
GGCTACGTGGACGACACGCTGTTCGTGAGGTTCGACAGCGACGCCACGA
GTCCGAGGAAGGAGCCGCGGGCGCCATGGATAGAGCAGGAGGGGCCGGA
GTATTGGGACCGGGAGACACAGATCTCCAAGACCAACACACAGACTTAC
CGAGAGAGCCTGCGGAACCTGCGCGGCTACTACAACCAGAGCGAGGCCG
GGTCTCACATCATCCAGAGGATGTATGGCTGCGACCTGGGGCCGGACGG
GCGCCTCCTCCGCGGGCATAACCAGTACGCCTACGACGGCAAAGATTAC
ATCGCCCTGAACGAGGACCTGAGCTCCTGGACCGCGGCGGACACCGCGG
CTCAGATCACCCAGCGCAAGTGGGAGGCGGCCCGTGAGGCGGAGCAGCT
GAGAGCCTACCTGGAGGGCCTGTGCGTGGAGTGGCTCCGCAGACACCTG
GAGAACGGGAAGGAGACGCTGCAGCGCGCGGACCCCCCAAAGACACACG
TGACCCACCACCCCATCTCTGACCATGAGGCCACCCTGAGGTGCTGGGC
CCTGGGCTTCTACCCTGCGGAGATCACACTGACCTGGCAGCGGGATGGC
GAGGACCAAACTCAGGACACTGA

An exemplary nucleic acid sequence encoding a human allo-MHC II can include a sequence as set forth in SEQ ID NO:2. Nucleic acid encoding a human MHC II polypeptide (e.g., an HLA-DP polypeptide, an HLA-DM polypeptide, an HLA-DOA polypeptide, an HLA-DOB polypeptide, an HLA-DQ polypeptide, or an HLA-DR polypeptide) can be included within a viral vector such that cells infected with the viral vector express the encoded MHC II polypeptide. In some cases, a nucleic acid sequence encoding a human allo-MHC II can be as described elsewhere (see, e.g., Robinson et al., 2005 Nucleic Acids Research 331:D523-526; and Robinson et al., 2013 Nucleic Acids Research 41:D1234-40).

SEQ ID NO: 2
ATGGTGTGTCTGAGGCTCCCTGGAGGCTCCTGCATGGCAGTTCTGACAG
TGACACTGATGGTGCTGAGCTCCCCACTGGCTTTGGCTGGGGACACCAG
ACCACGTTTCTTGGAGTACTCTACGGGTGAGTGTTATTTCTTCAATGGG
ACGGAGCGGGTGCGGTTACTGGAGAGACACTTCCATAACCAGGAGGAGC
TCCTGCGCTTCGACAGCGACGTGGGGGAGTTCCGGGCGGTGACGGAGCT
GGGGCGGCCTGTCGCCGAGTCCTGGAACAGCCAGAAGGACATCCTGGAA
GACAGGCGCGCCGCGGTGGACACCTATTGCAGACACAACTACGGGGCTG
TGGAGAGCTTCACAGTGCAGCGGCGAGTCCATCCTAAGGTGACTGTGTA
TCCTTCAAAGACCCAGCCCCTGCAGCACCACAACCTCCTGGTCTGTTCT
GTGAGTGGTTTCTATCCAGGCAGCATTGAAGTCAGGTGGTTCCGGAATG
GCCAGGAAGAGAAGACTGGGGTGGTGTCCACGGGCCTGATCCACAATGG
AGACTGGACCTTCCAGACCCTGGTGATGCTGGAAACAGTTCCTCGGAGT
GGAGAGGTTTACACCTGCCAAGTGGAGCACCCAAGCGTGACAAGCCCTC
TCACAGTGGAATGGAGAGCACGGTCTGAATCTGCACAGAGCAAGATGCT
GAGTGGAGTCGGGGGCTTTGTGCTGGGCCTGCTCTTCCTTGGGGCCGGG
CTGTTCATCTACTTCAGGAATCAGAAAGGACACTCTGGACTTCAGCCAA
GAGGATTCCTGAGCTGA

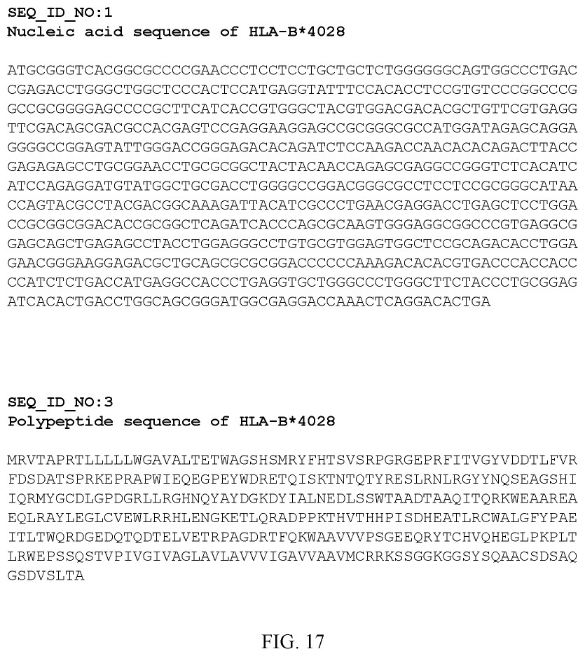

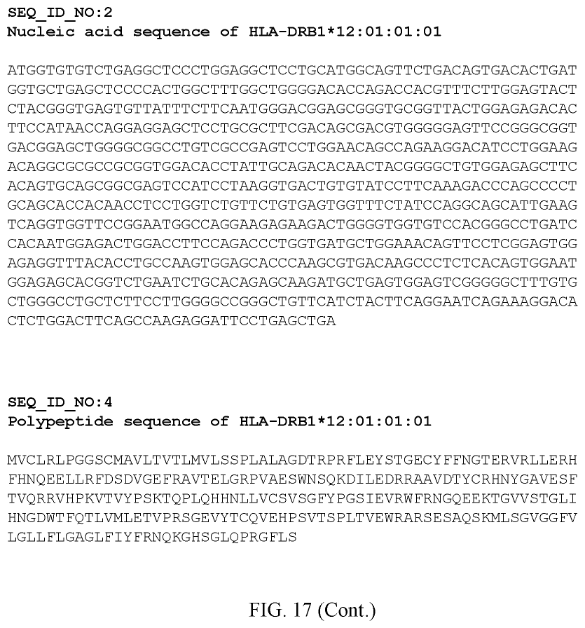

In some cases, a nucleic acid set forth in FIG. 17 can be included within a viral vector to express a human MHC I polypeptide, and that viral vector can be used to active naïve T cells within a mammal.

In some cases, a viral vector for activating naïve T cells in vivo as described herein can be designed to express a fragment of an MHC I polypeptide or a fragment of an MHC II polypeptide. A fragment of an MHC I polypeptide or an MHC II polypeptide can be from about 182 amino acids to about 273 amino acids (e.g., from about 182 amino acids to about 250 amino acids, from about 182 amino acids to about 225 amino acids, from about 182 amino acids to about 200 amino acids, from about 200 amino acids to about 273 amino acids, from about 225 amino acids to about 273 amino acids, from about 250 amino acids to about 273 amino acids, from about 190 amino acids to about 260 amino acids, from about 200 amino acids to about 250 amino acids, from about 215 amino acids to about 235 amino acids, from about 200 amino acids to about 220 amino acids, from about 220 amino acids to about 240 amino acids, from about 240 amino acids to about 260 amino acids, or from about 260 amino acids to about 280 amino acids) in length.

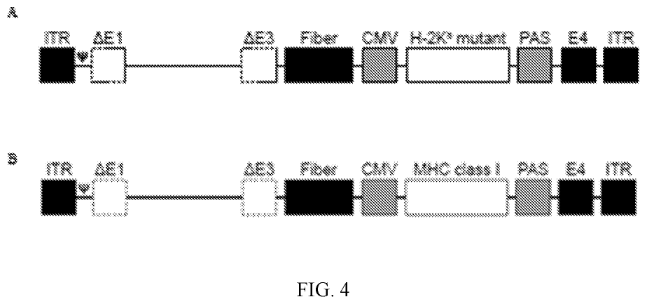

A viral vector for activating naïve T cells in vivo as described herein can be, or can be derived from, a viral vaccine. In some cases, a viral vector used as described herein can be replication-defective. In some cases, a viral vector used as described herein can be immunogenic. Examples of viral vectors that can be designed to encode an MHC class I or class II polypeptide and used to active T cells (e.g., naïve T cells) within a mammal include, without limitation, picomavirus vaccines, adenovirus vaccines, rhabdoviruses (e.g., vesicular stomatitis viruses (VSV)), paramyxoviruses, and lentiviruses. In some cases, naïve T cells described herein can be activated in vivo by administering to a human an immunogenic, replication-defective adenoviral vector encoding an allo-MHC I. An exemplary adenoviral vector encoding an allo-MHC I and/or allo-MHC-class II is shown in FIG. 4B.

This document also provides materials and methods for treating mammals (e.g., humans) having cancer (e.g., a cancer including cancer cells that express a tumor antigen). For example, naïve T cells described herein (e.g., naïve T cells expressing a tumor-specific antigen) can be activated in vivo to treat humans having cancer. In some cases, in vivo activation of naïve T cells as described herein can be used to reduce the number of cancer cells (e.g., cancer cells expressing a tumor antigen) within a mammal. In some cases, in vivo activation of naïve T cells as described herein can be used to slow and/or prevent recurrence of a cancer (e.g., a cancer in remission). In some cases, in vivo activation of naïve T cells as described herein can be used to target quiescent and/or non-dividing cancer cells (e.g., cancer cells expressing tumor antigens).

In some cases, the methods described herein for treating mammals (e.g., humans) having cancer can include identify the mammal as having cancer. Any appropriate method can be used to identify a mammal as having cancer. Once identified as having cancer, naïve T cells (e.g., naïve T cells obtained from the mammal having cancer) can be engineered (e.g., engineered in vitro or in vivo) to express antigen receptors (e.g., tumor-specific antigen receptors), and activated in vivo as described herein.

Any type of mammal having cancer can be treated using the materials and methods described herein. Examples of mammals that can be treated by in vivo activation of naïve T cells as described herein include, without limitation, primates (e.g., humans and monkeys), dogs, cats, horses, cows, pigs, sheep, rabbits, mice, and rats. For example, humans having cancer can be treated using in vivo activation of naïve T cells as described herein.

Any appropriate type of cancer can be treated using the materials and methods described herein. In some cases, a cancer to be treated as described herein can include one or more solid tumors. In some cases, a cancer to be treated as described herein can be a cancer in remission. In some cases, a cancer to be treated as described herein can include quiescent (e.g., dormant or non-dividing) cancer cells. In some cases, a cancer to be treated as described herein can be cancer that has escaped and/or has been non-responsive to chemotherapy. Examples of cancers that can be treated by in vivo activation of naïve T cells as described herein include, without limitation, leukemias (e.g., acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMOL)), lymphomas (e.g., Hodgkin's lymphomas and non-Hodgkin's lymphomas), myelomas, ovarian cancer, breast cancer, prostate cancer, colon cancer, germ cell tumors, hepatocellular carcinoma, bowel cancer, lung cancer, and melanoma (e.g., malignant melanoma).

The materials and methods described herein can be used to specifically target a cell (e.g., a cancer cell) expressing an antigen (e.g., a tumor antigen such as a tumor-specific antigen). For example, in vivo activation of naïve T cells as described herein can include engineering the naïve T cells to express a tumor-specific antigen receptor that can target (e.g., recognize and bind to) a tumor antigen. In some cases, a tumor antigen can be a cell surface tumor antigen. Examples of tumor antigens that can be targeted by in vivo activated T cells expressing a tumor-specific antigen receptor include, without limitation, MUC-1 (associated with breast cancer, multiple myeloma, colorectal cancer, and pancreatic cancer), HER-2 (associated with gastric cancer, salivary duct carcinomas, breast cancer, testicular cancer, and esophageal cancer), and ER (associated with breast cancer, ovarian cancer, colon cancer, prostate cancer, and endometrial cancer).

In cases where naïve T cells described herein (e.g., naïve T cells expressing tumor-specific antigen receptors) are engineered ex vivo to express a heterologous antigen receptor (e.g., a heterologous tumor-specific antigen receptor) as described herein and administered (e.g., by adoptive transfer) to a mammal (e.g., a human), any appropriate method can be used to administer the naïve T cells (e.g., engineered naïve T cells). Examples of methods of administering naïve T cells engineered to express a heterologous antigen receptor to a mammal can include, without limitation, injection (e.g., IV, ID, IM, or subcutaneous injection). For example, naïve T cells expressing tumor-specific antigen receptors can be administered to a human by IV injection.

In cases where naïve T cells described herein (e.g., naïve T cells expressing tumor-specific antigen receptors) are engineered ex vivo to express a heterologous antigen receptor (e.g., a heterologous tumor-specific antigen receptor) and administered (e.g., by adoptive transfer) to a mammal (e.g., a human), any appropriate number of naïve T cells (e.g., engineered naïve T cells) can be administered to a mammal (e.g., a mammal having cancer). In some cases, from about 200 naïve T cells described herein to about 1500 naïve T cells described herein (e.g., from about 200 naïve T cells to about 1300 naïve T cells, from about 200 naïve T cells to about 1250 naïve T cells, from about 200 naïve T cells to about 1000 naïve T cells, from about 200 naïve T cells to about 750 naïve T cells, from about 200 naïve T cells to about 500 naïve T cells, or from about 200 naïve T cells to about 400 naïve T cells) can be administered to a mammal (e.g., a human). For example, about 300 naïve T cells expressing tumor-specific antigen receptors can be administered to a human having cancer where the naïve T cells are then activated in vivo by allo-MHC I (e.g., allo-MHC I administered to the human having cancer using an immunogenic, replication-defective adenoviral vector encoding an allo-MHC I).

The invention will be further described in the following examples, which do not limit the scope of the invention described in the claims.

### EXAMPLES

### Example 1: Priming Cytotoxic T Cells (CTLs)

To examine if CTLs could be primed to hunt and kill quiescent cells expressing targetable antigens, 1200 OT-1 T cells were adoptively transferred into RIP-OVA mice (expressing the ovalbumin (OVA) antigen in pancreatic islets), and then activated with TMEV-OVA picomavirus vaccine.

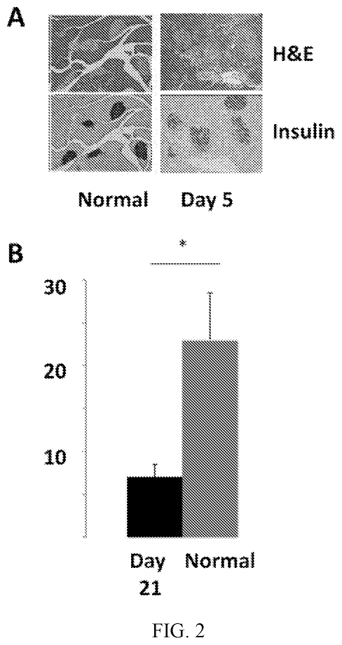

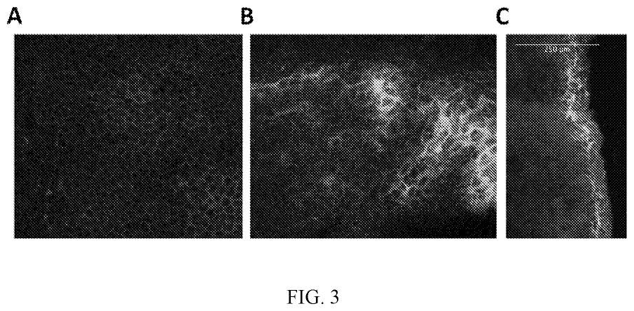

Pancreatic tissues were examined at using H&E staining and IHC staining for insulin. Pancreatic inflammation was seen within 5 days of CTL induction by virus (FIG. 2A). Significant destruction of islets was observed in surviving mice on day 21 (FIG. 2B). No virus was detected in the pancreas by PCR. As few as 300 naïve T cells activated in vivo by a picomavirus vaccine elicited complete destruction of normal virus free pancreatic islets within 10 days of activation. In contrast, 8×10⁷OT-1 spleen cells activated in donor mice and transferred into RIP-OVA mice were not pathogenic.

These results demonstrate that activated T cells can scan cells in the body for relevant antigens and elicit immune destruction in the absence of preexisting inflammation.

### Example 2: Activation of Allo-Reactive Cytotoxic T Cells (CTLs)

To determine whether adenovirus encoding allogeneic MHC I molecules can activate allo-reactive CTL, the allogeneic MHC class I gene was expressed in the context of an adenovirus infection into LN antigen presenting cells.

Adenovirus expressing Cre recombinase were introduced into the lymphatics of mTmG-reporter mice by intradermal injection. mTmG-reporter mice express a floxed membrane red fluorescent “tomato” and a silenced membrane GFP gene. In the presence of expressed cre, tomato is silenced and GFP is activated. Tomato expressing and GFP expressing T cells can be distinguished by fluorescent microscopy following introduction of an adenovirus expressing cre or a control adenovirus. Successful transduction results in the transition from red to green fluorescence. Cre recombinase was transduced in sub capsular sinus macrophage (FIGS. 3A-3C).

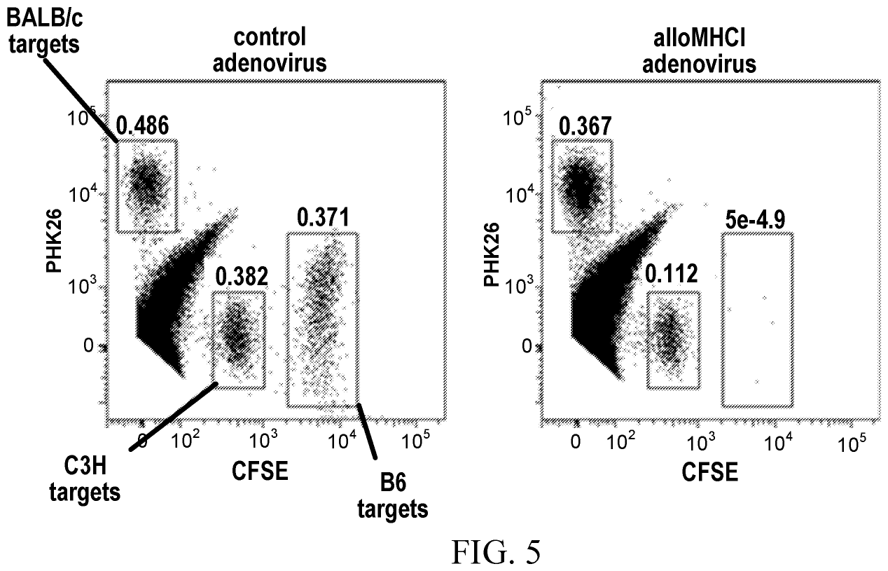

A replication-defective adenovirus (serotype 6) vector expressing a mutant MHC molecule which functions as a universal alloantigen (FIG. 4A) was introduced into LN by intradermal injection. Four days after introduction of adenovirus encoding allogeneic MHC I, syngeneic (BALB/c), allogeneic (B6), and third party (C3H) labeled target cells were adoptively transferred IV into challenged hosts in an in vivo CTL assay. Four hours later spleen cells were harvested and analyzed by flow cytometry for the presence of introduced target cells. B6 target cells (targets expressing introduced allo-MHC I) were completely eliminated in vivo. Potent allo-reactive CD8+ T cells were activated in just 4 days (FIG. 5).

These results demonstrate that intradermally injected adenovirus expressing allo-MHC I can present allo-MHC I antigen in sub capsular sinus macrophages and can activate CTLs that target cells expressing allo-MHC I.

### Example 3: Adoptive Transfer of Naïve Cytotoxic T Cells (CTLs)

To examine if adoptively transferred naïve CTL precursors migrate to secondary lymphoid organs and become activated by adeno-MHCI virus, allotype-marked naïve T cells were labeled with CFSE and adoptively transferred intravenously into naïve hosts which were subsequently challenged intradermally with adeno-MHCI to elicit an allo-reactive CTL response from the transferred cells.

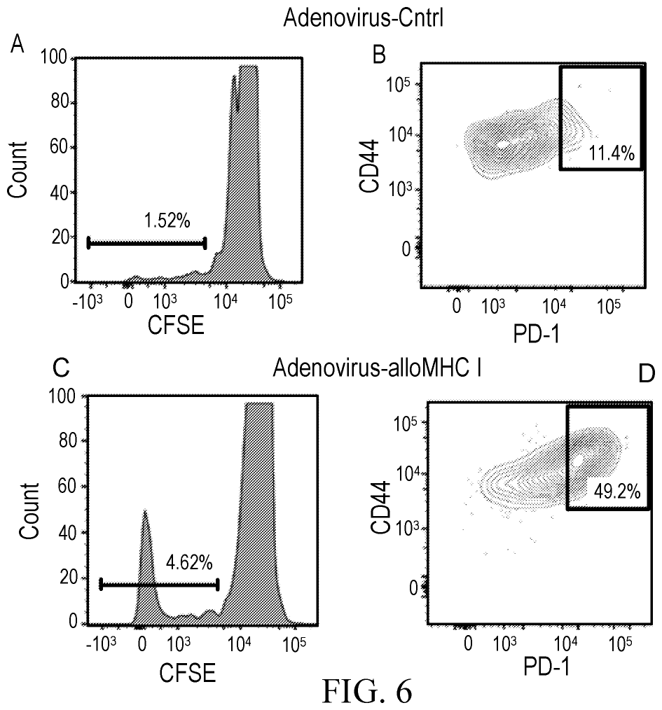

Adoptively transferred CFSE-labeled T cells migrated to the LN where they encountered and responded to transduced alloMHCI molecules (FIGS. 6A and 6C). Stimulated cells proliferated when stimulated with allo-MHCI, diluting the CFSE (FIG. 6C). The CFSE-dilute population displayed a more activated phenotype expressing high CD44 and PD-1 (FIG. 6D) relative to the CFSE-dilute cells isolated from lymph nodes challenged with control adenovirus (FIG. 6B). Approximately 4.5% of the transferred cells present on day 4 had proliferated (FIGS. 6A and 6C) and exhibited upregulation of activation markers (FIGS. 6B and 6D).

These results demonstrate that adoptively transferred CD8+ T cells can migrate to the LN and can be activated by an alloMHC I encoding adenovirus vaccine.

### Example 4: In Vivo Activation of Naïve Cytotoxic T Cells (CTLs)

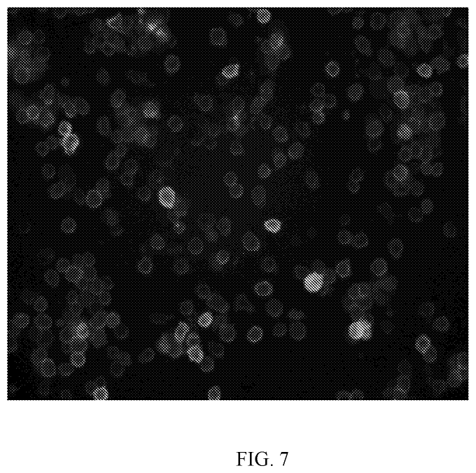

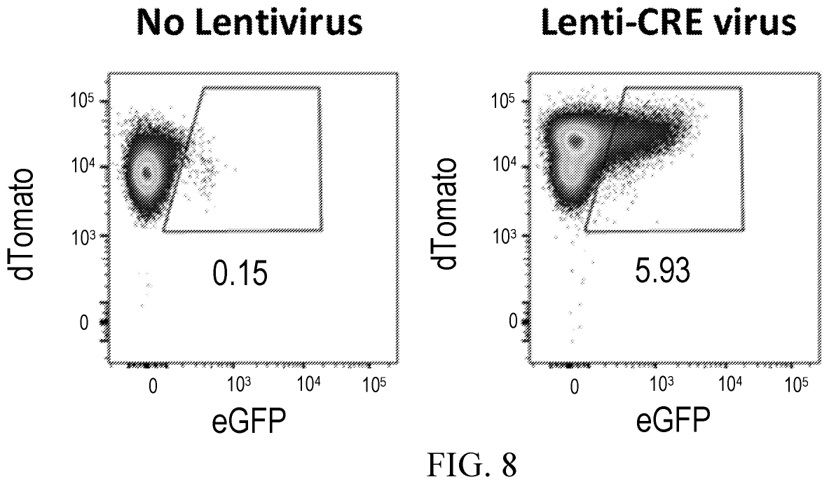

To examine whether in vivo activated T cells can migrate into tumors, an approach for evaluating the efficiency of viral transduction of T cells ex vivo was established using mTmG-reporter mice. Naïve CD8+ spleen cells from MTMG-reporter mice were transduced with lentivirus expressing cre by centrifugal concentration of virus and polybrene, and were subsequently activated with anti-CD3/CD28+IL-2 to maintain viability in culture for 4 days. Successful transduction results in the transition from red to green fluorescence (FIG. 7 and FIG. 8). Using the MTMG-reporter mouse, the efficiency of transfection, the migration of adoptively transferred T cells into lymph nodes, and migration of the adoptively transferred T cells into tumors can be determined.

### Example 5: In Vivo Activation of Naïve Cytotoxic T Cells (CTLs)

Humanized NSG (hu-NSG) mice with established human hematopoiesis provide a model for using lentivirus CAR to establish proof of concept. hu-NSG mice in donor matched batches with verified human leukocytes in circulation were obtained. These mice were used as donors of human cells for a CAR transduction scheme.

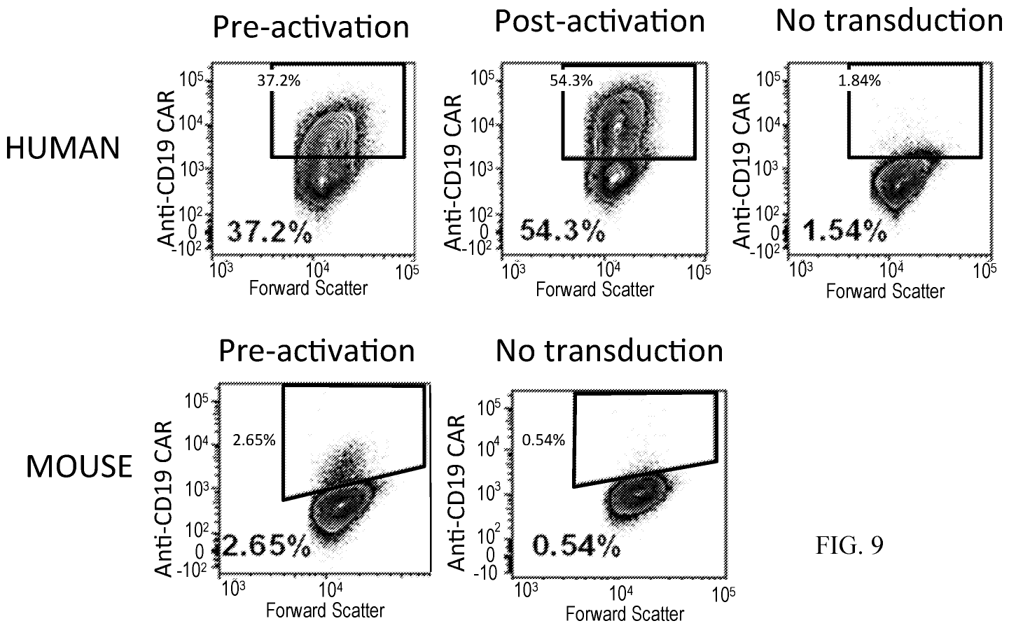

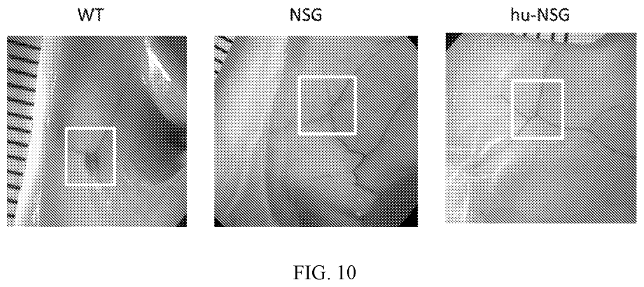

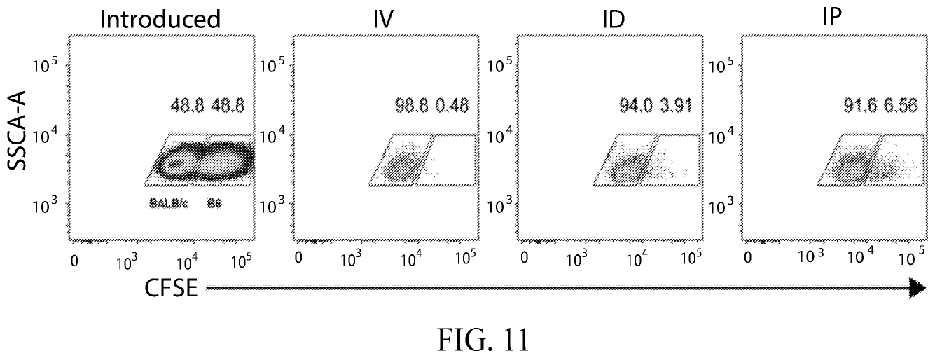

To determine if a CAR could activate CTLs in vivo, freshly isolated T cells were transduced with a lentiviral vector expressing human CAR19 (lenti-CAR19). Pooled CD4 & CD8 T cells transduced with lenti-CAR19 prior to or after activation (anti-CD3/CD28) and cultured 4 days to allow gene expression, then stained with anti-mouse antibody and analyzed using flow cytometry. Freshly isolated spleen cells were transduced with lenti-CAR19 for 1 hour and immediately transferred into syngeneic hu-NSG recipients (1 donor spleen/recipient). Mice also received Ad6-K^{b} vaccine at the time of cell transfer. Approximately 10% of the recovered human spleen cells were CAR+ in the three recipients. As shown in FIG. 9, human T cells were effectively transduced with lento-CAR19, but mouse cells were not.

Hu-NSG mice lack lymph nodes. The absence of lymph nodes in hu-NSG mice required a change in approach. To evaluate the effectiveness of intradermal introduction of non-replicating virus in hu-NSG mice, Evans blue was injected intradermally in the tail to mark inguinal lymph node in WT, NOD Scid IL-2Rγ^{-/-} (NSG), and human CD34+ hematopoietic cell reconstituted NSG mice (hu-NSG) (FIG. 10).

To determine if alternative routes of administration could be used for in vivo CTL, replication-defective adenoviral vectors encoding an allo-MHC I (Ad6-alloMHC (K^{b})) were delivered to hu-NSG mice multiple routes, and the ability to induce strong CTL activity was assessed. BALB/c mice received 10¹⁰ Ad6-K^{b} IV, ID, or IP. 1 week later, the mice received differentially labeled BALB/c (self) and B6 (alloMHC) target cells IV. Cells migrating into the spleen were assessed for both introduced populations. Effectiveness was indicated by the relative depletion of the B6 target cells. IV, ID, and IP vaccination were equally effective for inducing strong CTL activity (FIG. 11). In a subsequent experiment, mice received half the vaccine IV and half IP as the distribution and trafficking of human immune cells in the spleens and peritoneum of NSG mice is poorly defined.

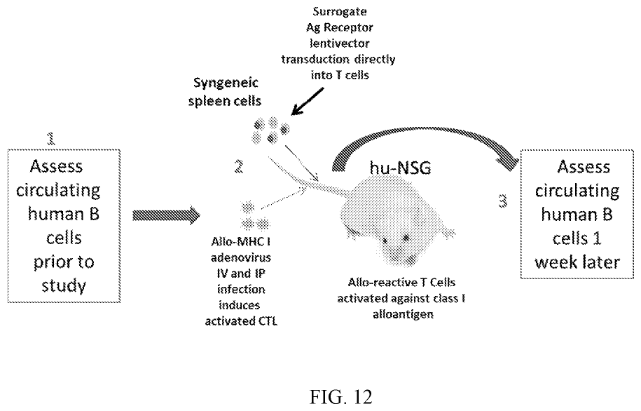

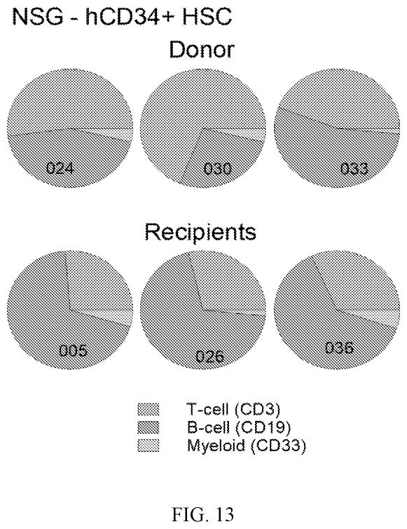

To determine whether adenovirus encoding allogeneic MHC I molecules can activate allo-reactive CTL to eliminated cells expressing a target antigen, hu-NSG mice were administered lentivirus-CAR19 transduced hu-NSG spleen cells and replication defective adenoviruses encoding the MHC allogeneic antigen H-2K^{b}. On overview of the method is shown in FIG. 12. Three hu-NSG mice with known T cell reconstitution were selected as lymphoid donors. The human leukocyte composition of hu-NSG mice selected as donors and hu-NSG mice selected as recipients are shown in FIG. 13. Spleen cells from donor animals were recovered, pooled, red cells lysed using ACK and then the whole product was suspended in 100 μL of undiluted lenti-CAR19 virus (MOI). Polybrene was added for final concentration of 8 μg/mL. The suspension was centrifuged at 800×g for 90 minutes at 31° C. The viral supernatant was removed, and the cell pellet was suspended in 300 μL PBS and injected IV (100 μL/mouse). 5×10⁹ viral particles of replication defective adenovirus 6 encoding the MHC allogeneic antigen H-2K^{b} was injected IV, and an identical dose was injected IP. The mice were monitored daily with no detrimental phenotypes observed for one week. On day 7, the mice were bled, and the composition of human B cells in the blood was assessed.

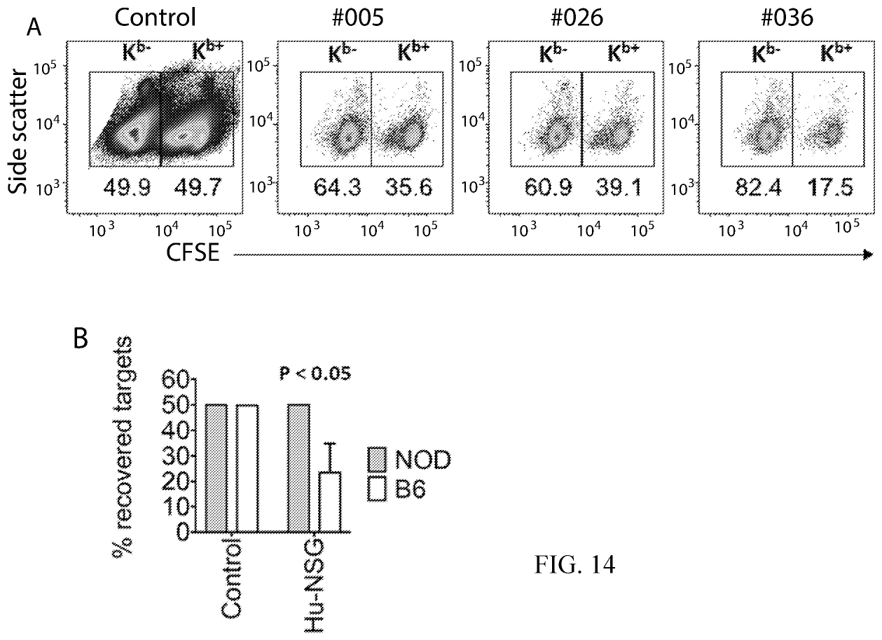

To determine if in vivo CTL induced anti-K^{b} cytotoxic activity in hu-NSG hosts, mice were challenged with a mixture of K^{b-} and K^{b+} target cells, and spleens of the recipient mice were examined. Four hours prior to harvesting blood and spleen cells from the hu-NSG recipients (which had received lentivirus-CAR19 transduced hu-NSG spleen cells and Ad6-H-2K^{b} vaccine 1 week earlier), mice were challenged with a 1:1 mixture of K^{b-} syngeneic NOD splenic target cells and K^{b+} allogeneic B6 splenic target cells differentially labeled with CFSE. Following the 4 hour in vivo incubation period, each of the spleens of the recipient mice was examined for the ratio of persisting labeled K^{b-} and K^{b+} target cells. The expected 1:1 ratio was altered in all three recipients indicating a preferential killing of the K^{b+} spleen cells (FIG. 14, panel A). The ratio of recovered K^{b+} cells was significantly lower relative to the K^{b-} target cells (FIG. 14, panel B). This analysis indicated CTL activity was induced by vaccination with Ad6-H-2K^{b} in the hu-NSG mice targeting K^{b} expressing cells.

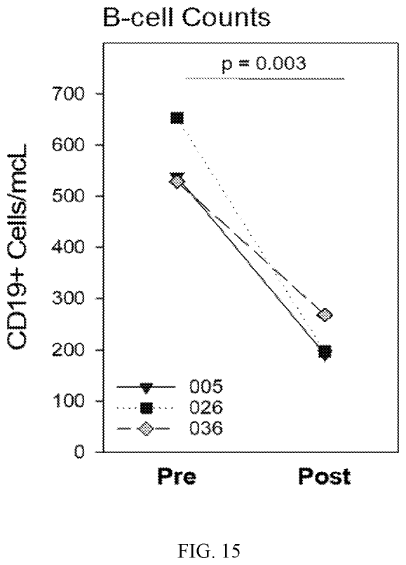

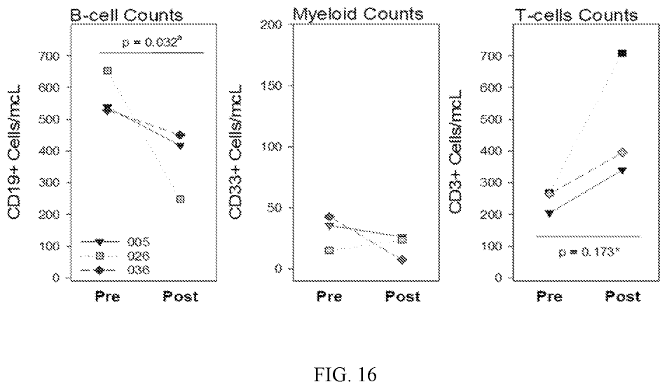

To determine if in vivo CTL activated against cells expressing a target antigen, human spleen cells from hu-NSG hosts that received CART treatment and AD6 vaccination were assessed for expression of the CAR19 protein. Raw data of the drop in B cell numbers in hu-NSG mice is shown in FIG. 15. The absolute numbers of recovered B cells pre and post therapy were highly significant. However, two variables could have contributed to this conclusion, non-specific depletion of peripheral blood cell populations, including B cells, by repeated blood sampling, and the intended effects of CAR T cell therapy. To confirm the drop in B cell numbers was due to CAR T cell therapy, data was normalized to remove possible non-specific depletion effects. The normalization of the post treatment values to the pretreatment values using the formula (total CD45+ cell counts pretreatment/CD45 cell counts post treatment×absolute counts of cell lineage+ cells post treatment) was a conservative approach, reducing the magnitude of observed differences between pre and post treatment values in the B cell compartment to account for no-specific depletion of B cells by repetitive sampling of the blood. One-tailed hypothesis testing used a paired T Test to reflect the hypothesis. The apparent increase in T cells following therapy is consistent with previous CART therapy findings. However, evaluation of this possibility was not part of the original hypothesis, therefore, a two tailed test was applied. The absence of change in myeloid counts suggests the observed drop in B cells and the apparent rise of T cells appears to be cell lineage specific (FIG. 16). There appears to be a correlation between the degree of B cell depletion and rise in T cells and in the level of CAR19 expression (FIG. 16, panel B) in the spleens of the recipient mice, associations also seen previously in CART therapy.

This analysis verified Ad6-K^{b} activated antigen-specific killing. The mice demonstrated activity against the CD19 target after administration of Ad6-MHC, as demonstrated by the depletion of circulating CD19+ B cells in the recipient mice.

These results demonstrate that naïve T cells expressing tumor-specific antigen receptors can be specifically activated (e.g., to become CTLs) in vivo by encountering a target antigen, and the in vivo activated T cells can target cells expressing the antigen.

### Example 6: Generation of Viral Vectors

To develop a viral vector encoding rare HLA class I molecules such as HLA-B*4028, partial nucleic acid sequences encoding exons 2 and 3 were obtained from publically available database (see, e.g., GenBank: AF181842 and AF181843, respectively; since replaced with AH008245.2) and were used to guide modification of the full-length coding sequence for HLA-B*4004 (see, e.g., GenBank: M84384.1) capable of producing a full-length HLA-B*4028 polypeptide (e.g., SEQ ID NO:3). To develop a viral vector also encoding rare HLA class II molecules such as HLA-DRB1*12:01:01:01, SEQ ID NO:2 was obtained from publically available data base (see, e.g., Robinson et al., 2005 Nucleic Acids Research 331:D523-526; and Robinson et al., 2013 Nucleic Acids Research 41:D1234-40), and used to produce a full-length HLA-DRB1*12:01:01:01 polypeptide (e.g., SEQ ID NO:4).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-67. (canceled)

68. A method for obtaining an activated T cell within a mammal, wherein said activated T cell comprises a heterologous antigen receptor, wherein said method comprises administering, to a mammal, (a) nucleic acid encoding said heterologous antigen receptor and (b) a virus comprising nucleic acid encoding an MHC class I polypeptide, wherein said nucleic acid is introduced into T cells within said mammal to form engineered T cells comprising said heterologous antigen receptor, wherein administration of said virus activates T cells within said mammal, and wherein at least one T cell within said mammal comprises said heterologous antigen receptor and is activated.

69. The method of claim 68, wherein said mammal is a human.

70. The method of claim 68, wherein said at least one T is a cytotoxic T lymphocyte.

71. The method of claim 68, wherein said antigen receptor is a chimeric antigen receptor.

72. The method of claim 68, wherein said antigen receptor is a tumor-specific antigen receptor.

73. The method of claim 68, wherein said nucleic acid encoding said heterologous antigen receptor is introduced into said T cells with a viral vector comprising said nucleic acid.

74. The method of claim 73, wherein said viral vector is a lentiviral vector or retroviral vector.

75. The method of claim 68, wherein said nucleic acid is administered to said mammal via intravenous injection.

76. The method of claim 68, wherein said nucleic acid is administered to said mammal via injection into a lymph node of said mammal.

77. The method of claim 68, wherein said virus is an adenovirus or a rhabdovirus.

78. The method of claim 68, wherein said virus is administered to said mammal via intradermal injection.

79. The method of claim 68, wherein said virus is administered to said mammal via direct administration into a lymph node of said mammal.

80. The method of claim 68, wherein said nucleic acid is administered to said mammal before said virus is administered to said mammal.

81. The method of claim 80, wherein said nucleic acid encoding said heterologous antigen receptor is introduced into said T cells with a lentiviral vector comprising said nucleic acid.

82. The method of claim 68, wherein said nucleic acid is administered to said mammal after said virus is administered to said mammal.

83. The method of claim 82, wherein said nucleic acid encoding said heterologous antigen receptor is introduced into said T cells with a retroviral vector comprising said nucleic acid.

84-85. (canceled)

86. The method of claim 68, wherein said at least one T cell comprises a native T cell receptor.

87. The method of claim 68, wherein said at least one T cell is a plurality of activated T cells comprising said heterologous antigen receptor.

88. The method of claim 87, wherein each of said activated T cells of said plurality of said activated T cells comprises a different native T cell receptor.

89-96. (canceled)


SEQUENCE LISTING (4 sequences)

SEQ ID NO: 1 - DNA, 758 bp, Homo sapiens
atgcgggtcacggcgccccgaaccctcctcctgctgctctggggggcagtggccctgaccgagacctgggctggctcccactccatgaggtatttccacacctccgtgtcccggcccggccgcggggagccccgcttcatcaccgtgggctacgtggacgacacgctgttcgtgaggttcgacagcgacgccacgagtccgaggaaggagccgcgggcgccatggatagagcaggaggggccggagtattgggaccgggagacacagatctccaagaccaacacacagacttaccgagagagcctgcggaacctgcgcggctactacaaccagagcgaggccgggtctcacatcatccagaggatgtatggctgcgacctggggccggacgggcgcctcctccgcgggcataaccagtacgcctacgacggcaaagattacatcgccctgaacgaggacctgagctcctggaccgcggcggacaccgcggctcagatcacccagcgcaagtgggaggcggcccgtgaggcggagcagctgagagcctacctggagggcctgtgcgtggagtggctccgcagacacctggagaacgggaaggagacgctgcagcgcgcggaccccccaaagacacacgtgacccaccaccccatctctgaccatgaggccaccctgaggtgctgggccctgggcttctaccctgcggagatcacactgacctggcagcgggatggcgaggaccaaactcaggacactga

SEQ ID NO: 2 - DNA, 801 bp, Homo sapiens
atggtgtgtctgaggctccctggaggctcctgcatggcagttctgacagtgacactgatggtgctgagctccccactggctttggctggggacaccagaccacgtttcttggagtactctacgggtgagtgttatttcttcaatgggacggagcgggtgcggttactggagagacacttccataaccaggaggagctcctgcgcttcgacagcgacgtgggggagttccgggcggtgacggagctggggcggcctgtcgccgagtcctggaacagccagaaggacatcctggaagacaggcgcgccgcggtggacacctattgcagacacaactacggggctgtggagagcttcacagtgcagcggcgagtccatcctaaggtgactgtgtatccttcaaagacccagcccctgcagcaccacaacctcctggtctgttctgtgagtggtttctatccaggcagcattgaagtcaggtggttccggaatggccaggaagagaagactggggtggtgtccacgggcctgatccacaatggagactggaccttccagaccctggtgatgctggaaacagttcctcggagtggagaggtttacacctgccaagtggagcacccaagcgtgacaagccctctcacagtggaatggagagcacggtctgaatctgcacagagcaagatgctgagtggagtcgggggctttgtgctgggcctgctcttccttggggccgggctgttcatctacttcaggaatcagaaaggacactctggacttcagccaagaggattcctgagctga

SEQ ID NO: 3 - protein, 362 aa, Homo sapiens
MRVTAPRTLLLLLWGAVALTETWAGSHSMRYFHTSVSRPGRGEPRFITVGYVDDTLFVRFDSDATSPRKEPRAPWIEQEGPEYWDRETQISKTNTQTYRESLRNLRGYYNQSEAGSHIIQRMYGCDLGPDGRLLRGHNQYAYDGKDYIALNEDLSSWTAADTAAQITQRKWEAAREAEQLRAYLEGLCVEWLRRHLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 4 - protein, 266 aa, Homo sapiens [sequence omitted]